(19) United States
(12) Patent Application Publication
(10) Pub. No.: US 2011/0071457 A1
(43) Pub. Date: Mar. 24, 2011

(54) Method and apparatus for controlling intracranial pressure

(75) Inventor: Kalyan Raman, Evanston, IL (US)

(73) Assignee: Northwestern University

(21) Appl. No.: 12/807,748

(22) Filed: Sep. 13, 2010

Related U.S. Application Data
(60) Provisional application No. 61/276,672, filed on Sep. 15, 2009.

(51) Int. Cl. A61M 1/00

(52) U.S. Cl. 604/9

(57) ABSTRACT

A regulator system and method are provided for controlling intracranial pressure (ICP) of a patient in a manner that permits continuous control of ICP to keep it on any clinically desired path over time. A nonlinear feedback control scheme for a CSF drainage valve embodies a relationship between intracranial pressure and infusion rate of CSF and an ICP state linearizer to keep the ICP on the clinically desired path over time.

### RELATED APPLICATION

This application claims benefits and priority of U.S. provisional application Ser. No. 61/276,672 filed Sep. 15, 2009, the entire disclosure of which is incorporated herein by reference.

### FIELD OF THE INVENTION

The present invention relates to a method and system for controlling intracranial pressure (ICP) using a feedback loop in a manner that permits control of ICP to keep it on any clinically desired path over time.

### BACKGROUND OF THE INVENTION

Excessive intracranial pressure (ICP) resulting from insufficient drainage of cerebrospinal fluid (CSF) leads to a neurological disorder called hydrocephalus. This disorder is evidenced by elevated cerebral spinal fluid (CSF), which can be due to excessive retention or production of CSF typically in the brain ventricles.

Hydrocephalus is treated by implanting a shunt to reduce ICP by draining excess CSF from the brain to another part of the body, such as the peritoneum or heart. Existing shunts are connected to valves which work like all-or-none devices. Opening these valves induces drainage of cerebrospinal fluid (CSF) through the shunt at a high rate and closing them shuts off drainage completely. These on-off shunts function like simple on-off switches and lack capability of continuous controlled CSF drainage over time, especially to provide continuous regulation of the patient's ICP to keep it on the clinically desired path over time.

### SUMMARY OF THE INVENTION

The present invention provides a regulator system and method for controlling intracranial pressure (ICP) of a patient in a manner that permits control of ICP to keep it on any clinically desired path over time and involving, in a feedback loop, measuring actual ICP at a given time, comparing the actual ICP and a desired ICP, and controlling drainage in response to the difference between the actual ICP and the desired ICP at a given time in feedback loop manner wherein ICP is adjusted to account for CSF infusion. In an illustrative embodiment, the present invention controls ICP using nonlinear feedback control for a CSF drainage valve wherein the control scheme embodies a relationship expressing the influence of CSF infusion on ICP and employs an ICP state linearizer to keep the ICP on the clinically desired path over time.

In another illustrative embodiment of the invention, the regulator system and method pursuant to the invention monitor and control the ICP to keep it at safe levels at all times for a patient suffering from hydrocephalus. The system and method are implemented through a well-defined and precisely executable algorithm control scheme that is incorporated in the control scheme of the feedback loop for the CSF drainage valve wherein the algorithm control scheme embodies a relationship of CSF infusion rate influence on ICP and employs an ICP state linearizer to bring the ICP back on track to any pre-defined clinically desired path or trajectory at all times at a rate determined by a tuning factor of the linearizer. Valve drainage action is thereby controlled by the nonlinear feedback control scheme.

Advantages and features of the present invention will become more readily apparent from the following detailed description taken with the following drawings.

### BRIEF DESCRIPTION OF THE DRAWINGS

FIG. 1 schematically illustrates a CSF shunt system implanted in a patient to drain CSF from a brain ventricle to the peritoneal cavity of the patient

FIG. 2 is a block diagram that illustrates the overall control logic and implementation of nonlinear feedback control of the ICP regulator system pursuant to an embodiment of the invention.

FIG. 3 is a block diagram that illustrates a particular control logic and implementation of nonlinear feedback control of the ICP regulator system pursuant to an embodiment of the invention.

FIG. 4 schematically illustrates implementation of FIG. 2.

FIG. 5 is a block diagram that illustrates an engineering equivalent of FIG. 4.

### DETAILED DESCRIPTION OF THE INVENTION

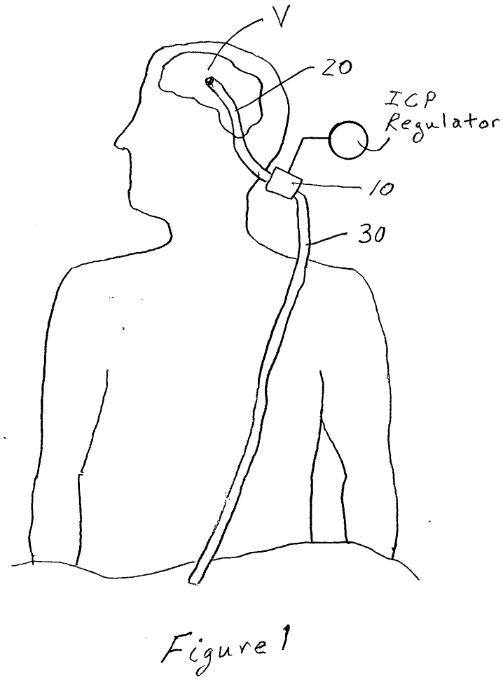

The regulator system and method pursuant to an illustrative embodiment of the invention monitor and control the patient's ICP over time as implemented through a well-defined and precisely executable algorithm control scheme that is incorporated in a microprocessor of a feedback loop for control of a CSF drainage valve 10 that is part of CSF drainage path. FIG. 1 illustrates schematically such a CSF drainage path having a drain catheter 20 that is implanted in the brain of a patient suffering from hydrocephalus to drain excess CSF via valve 10 to another part of the patient's body, such as the peritoneal cavity or heart, via a second discharge catheter 30 as is well known. The catheter 20 usually is located to drain CSF from the ventricles of the patient's brain as is also well known.

In practice of an embodiment of the present invention, control of the patient's ICP continuously over time is implemented through a well-defined and precisely executable algorithm control scheme that is incorporated in the microprocessor that is part of the feedback loop for the CSF drainage valve 10. This is in contrast to use of on-off type drainage valve used heretofore in such system to treat hydrocephalus disorder.

Pursuant to an illustrative embodiment of the invention, the algorithm control scheme embodies a relationship expressing the influence of CSF infusion rate on ICP and employs an ICP state linearizer to bring the ICP back on track to clinically desired path or trajectory at all times at a rate determined by a tuning factor of the linearizer. The nonlinear controller described herein is the first to provide a continuous feedback regulator for ICP. In the past, CSF infusion rate has been preset to be 1.5 ml/min in pressure-volume compensation studies conducted at Addenbrooke's Hospital, Cambridge, UK, by the University of Cambridge Neuroscience group in England, UK (Czosnyka et al. 2004). It also appears to have been a fairly standard infusion rate employed in past studies conducted elsewhere. The objective of past infusion studies was to understand the hydrodynamics of CSF flow as a function of the amount of CSF fluid let into or remaining in the cerebral cavity such that the studies have implications for the treatment of hydrocephalus. However, the standard rate of 1.5 ml/min used in the past studies was like an open-loop control and not a closed-loop or feedback control which can be correlated with the valve action to optimize the shunt performance on a continuous basis and implementable as part of the control algorithm as described below. Valve drainage action pursuant to the invention is thereby controlled by the nonlinear feedback control scheme using mathematical control theory of nonlinear systems in a manner to achieve continuous CSF drainage to keep ICP at safe clinical levels at all times.

Practice of an illustrative embodiment of the invention exploits and embodies a relationship between ICP (intracranial pressure) and infusion rate of CSF in a manner described below and that is enabled by a nonlinear feedback control of the CSF drainage valve 10.

Referring to FIG. 2, a simplified schematic block diagram of apparatus pursuant to an embodiment of the invention is shown to illustrate the overall control logic of the ICP regulator system and method. In particular, an illustrative embodiment of the present invention involves the boxes labeled ICP Regulator and CSF Process Dynamics. The logic underlying those boxes is described mathematically below. The mathematical formula provides an algorithm for continuous regulation of the patient's ICP to keep it on the clinically desired path.

The method for controlling intracranial pressure (ICP) in a patient involves a feedback loop, FIG. 2, including the steps of measuring actual ICP at a given time, comparing the actual ICP and a desired ICP, and controlling drainage in response to the difference between the actual ICP and the desired ICP at a given time in the feedback loop. The step of controlling drainage in response to the difference between actual ICP and desired ICP at a given time preferably involves adjusting the difference in actual (measured) ICP and desired ICP to account for the CSF infusion rate effect on ICP as described below and employing a ICP state linearizer as described below to maintain the ICP close to or on a desired clinical path.

To make the connection between the block diagram of FIG. 2 and the mathematical algorithms explicit, we link the terms in the boxes to the mathematics of Theorems 1 and 2 which constitute the algorithm and its properties. Theorem 1 contains the key results for the functional design of the ICP regulator (controller). Theorem 2 extends Theorem 1 to deal with the practical issue of noise and disturbances that contaminate all physical and biological processes in the real world. Theorem 2 provides a guarantee that the ICP regulator system design developed mathematically in Theorem 1 will function well under real-world conditions. Theorem 2 provides a firm, rigorous and compelling mathematical basis for the functioning of the system. Thus, the content of Theorem 2 provides precise information about system reliability that goes well beyond the mathematical level and depth of the analysis that is usually conducted at this stage.

Desired ICP: This is denoted by p_{d}(t) in the mathematics of the theorems
Comparison: This is accomplished by computing the difference between p(t) and p_{d}(t), where p(t) is the intracranial pressure (ICP)
ICP Regulator: This is the formula shown in Theorem 1 for I(t) and v(t). These two quantities provide the basis for the new control valve design operation (control scheme).
CSF Process Dynamics: This is described by the differential equation for p(t) in Theorem 1 and by the stochastic differential equation in Theorem 2
Output: The output is the ICP p(t) value (signal) provided to drainage valve 10
Measurement of ICP: This is the level of p(t) measured by an ICP sensor

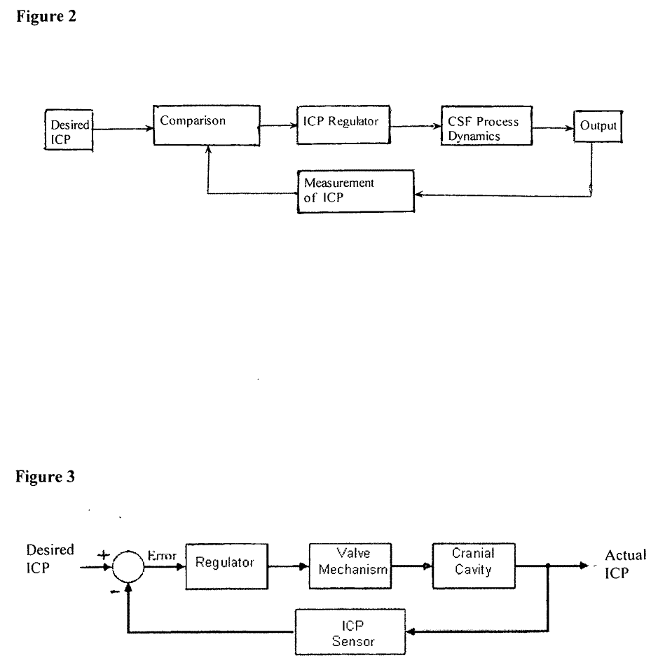

FIG. 3 is a block diagram that illustrates a particular control logic and implementation of nonlinear feedback control of the ICP regulator system pursuant to an embodiment of the invention based on FIG. 2. In FIG. 3, the circle with + and − symbols represents the Comparison block of FIG. 2 where the measured ICP p(t) is compared with the desired ICP to generate an ICP difference value (error signal) which difference value is provided to the Regulator block. In FIG. 2, the Regulator block includes the algorithmic features of the ICP controller where the difference value is adjusted for the CSF infusion rate effect by the state linearizer. The Regulator block can comprise a digital controller such as a microprocessor having the algorithm described below in detail below programmed in its software and also can include a comparator to carry out the Comparison step. The Valve Mechanism block corresponds to the drainage valve 10, which is controlled by signals, correlated with the optimized infusion rate, from the microprocessor of the preceding block to control CSF drainage of the cranial cavity of the patient. The ICP p(t) is determined by the algorithm in which I(t) influences the dynamic evolution of p(t) at the Output block. That is, the algorithm accounts for the influence by the production, storage, and re-absorption of the CSF on ICP. In FIG. 3, the Cranial Cavity block represents the cerebral space.

In FIG. 3, the actual ICP is measured using ICP sensor, which provides input values (signals) to the comparator of the Comparison step.

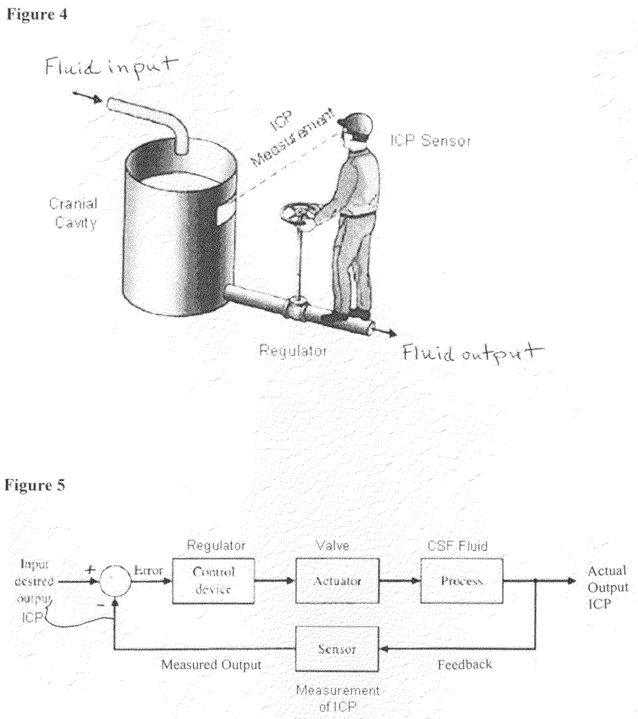

The details of implementation of the regulator system can vary widely. For purposes of illustration and not limitation, the actual ICP can be measured by a conventional ICP sensing or monitoring system such as including, but not limited to, an ICP monitoring system manufactured by Codman & Shurtell, Inc., a Johnson & Johnson company, Raynham, Mass., that uses a CODMAN MICROSENSOR (transducer) to sense ICP in an intraparenchymal sensing mode, a pressure transducer-tipped catheter to sense ICP in an intraventricular sensing mode, a ICP sensor system using an external ICP sensor, and any other suitable ICP sensor. The digital controller typically comprises a microprocessor that can include a comparator and that has the algorithm described below in detail programmed in its software. The drainage valve 10 is controlled by the microprocessor and can comprise a pressure regulating solenoid valve that opens/closes in response to command signals from the Regulator block as needed to regulate CSF drainage and thus ICP of the patient. The drainage valve 10 can be controlled and actuated by the series of microprocessor command signals (from the Regulator block) to drain CSF in a manner to maintain the patient's ICP close to or on the clinically desired path or trajectory at all times. Valve drainage action is thereby controlled by the nonlinear feedback loop control scheme using mathematical control theory of nonlinear systems in a manner to achieve continuous CSF drainage to keep ICP at safe clinical levels at all times as schematically shown in FIG. 4. The regulator system can be powered by battery and/or building power depending on usage.

FIG. 5 shows a regulator system similar to that of FIG. 3 with different labels of the components wherein the Regulator is labeled as Control Device (e.g. microprocessor), wherein the Valve is labeled Actuator (e.g. pressure regulating valve) and wherein CSF Fluid is labeled Process and represents its hydrodynamics. The actual ICP is measured by Sensor to provide the Measured output signal to the comparator represented as in FIG. 3. The values of the desired ICP, actual ICP, and measured ICP can be provided from the feedback loop to a data acquisition system such as including, but not limited to, a visual display for viewing of the values, a printer for printing the values for viewing, and/or to a computer system memory for storage and use of the values.

### The Mathematics of a Nonlinear Regulator for Controlling ICP

The Marmarou equation (Marmarou 1973, Marmarou 1978, Raman 2009, Raman 2008 listed below, all incorporated herein by reference) describes the pressure-volume compensation relationship that characterizes the circulatory dynamics of CSF. The notation is as follows—p(t) is the ICP at time “t,” R is the resistance to CSF outflow and E is the cerebral elasticity (Czosnyka 2004), p_{b} is a baseline pressure and I(t) is the infusion rate in the cranial cavity at time “t” which can be expressed as ml/min, although other units of measure can be used. The units of measure of the terms in the equation set forth herein are selected accordingly. The Marmarou model is the following nonlinear ordinary differential equation.

$$\frac{d p}{d t} = - \frac{E}{R} {p}^{2} + \frac{E}{R} {p}_{b} + EpI ( t )$$

Let

$$α = \frac{E}{R} ;$$

and rewrite the above equation as shown:

$$\frac{d p}{d t} = α ( {p}_{b} + RI ( t ) p - {p}^{2} )$$

Let p_{d}(t) be the clinically desirable trajectory for the ICP at time “t.” In particular, p_{d}(t) could be a constant level of pressure, say p_{d}(t)≡p_{c}, but this need not necessarily be the case.

### Theorem 1

At time “t,” choose I(t) (infusion rate of CSF related to circulatory dynamics of CSF production, storage, and re-absorption) as a feedback control relating it to the ICP p(t) as follows:

$$I ( t ) = \frac{p ( t )}{R} + \frac{\{ \frac{v ( t )}{E} - \frac{{p}_{b}}{R} \}}{p ( t )}$$

where v(t) (called ICP state linearizer) is chosen as follows:

$$v ( t ) = \frac{d {p}_{d}}{d t} - λ ( p ( t ) - {p}_{d} ( t ) )$$

In the above definition of v(t), λ is a tuning parameter which may be chosen arbitrarily to regulate the speed of correction of deviations of the ICP from the desirable trajectory for the ICP at time “t.” A large value of λ will regulate the ICP more quickly, bringing the ICP back on track into the clinically desirable trajectory at a faster rate. As a special case, if a constant level of pressure is desired at all times, say p_{d}(t)≡p_{c}, then

$$\frac{d {p}_{d}}{d t} = 0 ,$$

and under these circumstances, v(t) is chosen more simply as follows:

v(t)=−λ(p(t)−p_{d}(t))

Under these conditions, the ICP at any given time will stay exactly on the clinically desirable trajectory for all future times if it starts on it at the initial time; and if the ICP is not initially on the clinically desirable trajectory, then it will converge to it exponentially fast, thereby assuring rapid regulation of the patient's ICP.

Thus, the values of I(t) (expressing relationship of ICP and infusion rate of CSF) and v(t) (state linearizer) provide a nonlinear controller function to achieve the clinical objective of continuously regulating the ISCP so it stays close to or on the desired trajectory. The ICP regulator block provides an input value of I(t) to the CSF Process Dynamics block equation to calculate p(t) wherein each input value of I(t) is determined using the previously measured value of p(t)−p_{d}(t). By the very nature of differential equations, only the previously (most recently) measured value of p(t) will influence the future evolution of p(t). The ICP p(t) is thus determined by a nonlinear differential equation in which I(t) influences the dynamic evolution of p(t).

### Proof

The ICP at any time “t” is related to the infusion rate I(t) by the nonlinear ordinary differential equation (ODE)

$$\frac{d p}{d t} = α ( {p}_{b} + RI ( t ) p - {p}^{2} )$$

Substituting the feedback control

$$I ( t ) = \frac{p ( t )}{R} + \frac{\{ \frac{v ( t )}{E} - \frac{{p}_{b}}{R} \}}{p ( t )}$$

into the above ODE transforms the dynamics of the pressure-volume compensation into a new ODE.

$$\begin{matrix}\frac{d p}{d t} = α ( {p}_{b} + RI ( t ) p - {p}^{2} ) \\ = α ( {p}_{b} + R [ \frac{p ( t )}{R} + \frac{\{ \frac{v ( t )}{E} - \frac{{p}_{b}}{R} \}}{p ( t )} ] p - {p}^{2} ) \\ = α ( {p}_{b} + R [ \frac{p ( t )}{R} + \frac{\{ \frac{v ( t )}{E} - \frac{{p}_{b}}{R} \}}{p ( t )} ] p - {p}^{2} ) \\ = α ( {p}_{b} + {p}^{2} + \frac{v ( t )}{α} - {p}_{b} - {p}^{2} ) \\ = v ( t )\end{matrix}$$

Next, by choosing

$$v ( t ) = \frac{d {p}_{d}}{d t} - λ ( p ( t ) - {p}_{d} ( t ) ) ,$$

the dynamics of the new ODE is transformed again into the final dynamics we need to ensure tracking the ICP along the clinically desirable path p_{d}(t) as follows.

$$\frac{d p}{d t} = \frac{d {p}_{d}}{d t} - λ ( p ( t ) - {p}_{d} ( t ) )$$

Finally define the tracking error ε(t)=p(t)−p_{d}(t); then, upon rewriting the above ODE by transferring the term

$$\frac{d {p}_{d}}{d t}$$

to the left hand side of the equation, it is clear that the tracking error dynamics finally satisfies the ODE

$$\frac{d ɛ}{d t} = - λɛ ( t )$$

Studying the final dynamics resulting from the choice of feedback control, we can conclude the following:
(1) If the ICP is initially exactly as desired, then the tracking error at time zero is zero, ε(0)=0, and, therefore from

$$\frac{d ɛ}{d t} = - λɛ ( t ) ,$$

it follows that ε(t)=ε(0)e^{-λt}, and from that it follows that the tracking error will always remain zero at all future times.
(2) If the ICP is initially higher than desired, p(t)>p_{d}(t) at time 0, then the tracking error at time zero is positive, ε(0)>0, and, from ε(t)=ε(0)e^{-λt}, it follows that the tracking error will converge exponentially fast to zero.
(3) If the ICP is initially lower than desired, p(t)<p_{d}(t) at time 0, then the tracking error at time zero is negative, ε(0)<0, and, from ε(t)=ε(0)e^{-λt}, it follows that the tracking error will converge exponentially fast to zero.
(4) The above conclusions hold for conditions starting at any time ‘t₀’ not just the initial time t=0. This is because the solution to the tracking error dynamics

$$\frac{d ɛ}{d t} = - λɛ ( t ) ,$$

starting at any arbitrary time ‘t₀’ is ε(t)=ε(t₀)e^{-λ(t-t}^{₀}⁾, and the behavior of this solution is identical to that of ε(t)=ε(0)e^{-λt}, which corresponds to starting the dynamics at t₀=0.

While λ may theoretically be chosen as large or small as desired, in actual medical applications its value will be a function of the properties of the available materials for valve construction and the engineering design used in implementing the valve.

### Real-World Considerations

All mathematical models are approximations of reality, and the ICP regulator is based upon a mathematical model of the pressure-volume compensation model that describes the circulatory dynamics of CSF production, storage and reabsorption. In practice, disturbances due to uncontrolled factors may affect the circulatory dynamics, and such disturbances are not incorporated into the Marmarou model. Therefore, we must ask—how well will the regulator perform in practice, given that the mathematical model embodied in the Marmarou equation may not be an exact description of how the ICP evolves over time? Theorem 2 answers this question by generalizing the Marmarou model to incorporate the disturbances due to uncontrolled factors that are typical of real-world phenomena, and then studying how the ICP regulator performs under those conditions. Theorem 2 shows that, even under the influence of uncontrolled disturbances, the regulator will behave on average in the same way as described in Theorem 1. In other words, the regulator is robust with respect to disturbances on average.

### How Disturbances Due to Uncontrolled Factors Affect the Regulator

To study this, we need to incorporate disturbances in the Marmarou model because the regulator is based upon that model. For processes unfolding continuously in time, the standard and well-accepted way of doing this is to represent the effect of all disturbances by Brownian Motion. Brownian Motion provides a source of noise for continuous-time processes such as the pressure-volume compensation process involved in CSF dynamics. Brownian Motion is denoted by the symbol W(t). It satisfies the following properties:
(A) W(t) has independent increments over non-overlapping time intervals
(B) W(t)−W(s) has a Gaussian distribution over the time interval [s, t] with mean zero and variance σ²(t−s).

The Marmarou model may now be generalized to include the effect of disturbances by using W(t) as a noise source driving the deterministic differential equation

$$\frac{d p}{d t} = α ( {p}_{b} + RI ( t ) p - {p}^{2} ) .$$

This results in the following stochastic differential equation (SDE) (Raman 2009, Raman 2008 incorporated herein by reference):

dp=α(pₕ+RI(t)p−p²)dt+σpdW

The original Marmarou model which incorporates no disturbances is a special case corresponding to σ=0 (this means that the intensity of the noise is zero). The SDE is more realistic because it allows for the effect of noise on CSF dynamic evolution. An intuitively appealing and useful way of studying the effect of noise on the ICP regulator is to analyze its average performance. This is consistent with the standard treatment of random phenomena in both engineering and statistical practice—we study how a system under the influence of noise behaves on average. Here, our interest centers on the following critical question—will the regulator keep the ICP on the clinically desirable track on average? Theorem 2 answers this key question affirmatively.

### Theorem 2

The regulator developed in Theorem 1 will keep the average ICP at any given time exactly on the clinically desirable trajectory for all future times if it starts on it at the initial time; and if the ICP is not initially on the clinically desirable trajectory, then the average ICP will converge to it exponentially fast, thereby assuring rapid regulation of the patient's ICP on average.

### Proof

Consider the SDE dp=α(pₕ+RI(t)p−p²)dt+σp dW; upon substituting the expression for the regulator for I(t), it is transformed into the new SDE:

dp=(p_{d}′(t)+λp_{d}(t)−λp)dt+σpdW

In the above SDE, p_{d}′(t) denotes the time derivative

$$\frac{d {p}_{d}}{d t}$$

of the clinically desirable trajectory.

The above SDE may be re-written as:

d[p−p_{d}]=(−λ(p−p_{d}(t))dt+σpdW

Define the tracking error ε(t)=p(t)−p_{d}(t) and run the expectation operator E through each side of the above equation:

E[d[ε(t)]=E(−λ(p−p_{d}(t))dt+E[σpdW]

Fubini's Theorem allows interchanging the expectation and differentiation operators and we shall do so. This results in the following ODE for the average tracking error E[ε(t)]:

d[E[[ε(t)]]=(−λE(p−p_{d}(t)))dt+E[σpdW]

But, by the rules of Ito calculus, the term E[σp dW]=0; therefore we conclude

$$\frac{d [ E ( ɛ ( t ) ) ]}{d t} = ( - λ E ( ɛ ( t ) )$$

We see that, under the influence of uncontrollable disturbances, the average tracking error E[ε(t)] satisfies the same ODE as the tracking error ε(t) does under the effect of no disturbances. This immediately shows that all the conclusions of Theorem 1 also apply to the average performance of the regulator in the more realistic case—which will be encountered in real-world practice—in which the regulator is affected by uncontrolled disturbances.

The present invention will have many expected commercial applications as a result of the non-linear feedback control system which will improve shunts used in the treatment of hydrocephalus. Currently there are no cures for hydrocephalus and the only treatment is through the implantation of shunts but basic shunt technology has not changed much over time. Thus, the potential benefits of designing a better shunt are extremely high. The invention embodies nonlinear control theory in conjunction with the mathematics of stochastic differential equations to develop a rigorous foundation for more effective shunts based on a feedback regulator.

The present invention is not limited to the particular illustrative embodiments described in detail above. It is important to recognize that these embodiments are not the only or exclusive way to maintain the ICP on any clinically desired path at all times. Practice of the invention recognizes that there are a variety of alternative ways to achieve regulation of the ICP on a desired path. For example, in the implementation of the algorithm, infinitely many values are possible for the tuning parameter λ which controls the speed of convergence of the ICP back to the clinically ideal level from an undesirable level. Furthermore, the above nonlinear controller is based on the standard model of cerebrospinal fluid dynamics in which the reference pressure is assumed to be zero. Some researchers advocate a non-zero level for the reference pressure. The above-described algorithm also covers that case and can be mathematically tweaked to accommodate the non-zero reference pressure case should that be desired under certain circumstances. Finally, by their very nature, it is noted that non-linear problems rarely have a unique solution. The mathematics of non-linear control is complicated and every non-linear control problem must be analyzed on its own merit—there are no text-book solutions or cookbook recipes that solve every possible non-linear control problem. Thus, it is also possible to take alternative methodological approaches to the development of the algorithm—for example, it may be possible to design an algorithm that achieves similar results as the ICP system regulator by using robust control, adaptive control, sliding control, stochastic or deterministic optimal control, or even linear control in which the linear controller is an approximation of the original non-linear problem. The non-linear controller algorithm description recognizes that all these competing alternatives are encompassed and envisioned by the present invention.

Thus, although the invention has been described in connection with certain illustrative embodiments thereof, those skilled in the art will appreciate that changes and modifications can be made thereto within the scope of the invention as set forth in the following claims.

### REFERENCES

### Which are Incorporated Herein by Reference

Czosnyka, Marek; Czosnyka, Zofia, Momjian, Shahan and Pickard, John D.: Cerebrospinal Fluid Dynamics, Physiological Measurement 2004, 25: R51-R76.
Marmarou A (1973), “A theoretical model and experimental evaluation of the cerebrospinal fluid system,” Thesis, Drexel University, Philadelphia, Pa.
Marmarou A, Shulman K, Rosende R. M. (1978), “A nonlinear analysis of the cerebrospinal fluid system and intracranial pressure dynamics,” J Neurosurg, 48, 332-344.
“A Brownian Motion Model of Cerebrospinal Fluid Dynamics,” Kalyan Raman, Paper 23, SRHSB Proceedings 2008, 52^{nd} Annual Scientific Meeting, Brown University, Providence, R.I.
“Modeling, Estimation and Optimal Control Issues in Cerebrospinal Fluid Dynamics,” Kalyan Raman, Cerebrospinal Fluid Research 2009, 6 (Suppl 2), S23, 27^{th} November.

1. A method for controlling intracranial pressure (ICP) in a patient using a drainage path for cerebral spinal fluid (CSF), comprising in a feedback loop, measuring actual ICP at a given time, comparing the actual ICP and a desired ICP, and controlling CSF drainage in response to the difference between the actual ICP and the desired ICP wherein ICP is adjusted to account for CSF infusion.

2. The method of claim 1 further including controlling drainage using an ICP state linearizer to maintain the ICP close to or on a desired clinical path.

3. Apparatus for controlling intracranial pressure (ICP) in a patient using a drainage path for cerebral spinal fluid (CSF), comprising a sensor for measuring actual ICP at a given time, a comparator for comparing the actual ICP and a desired ICP, and a controller for controlling drainage in response to the difference between the actual ICP and the desired ICP at a given time in a feedback loop wherein the controller adjusts ICP to account for CSF infusion.

4. Apparatus of claim 3 wherein the controller includes an algorithm control scheme for controlling drainage in response to the difference between actual ICP and desired ICP at a given time adjusted to account for CSF infusion rate.

5. Apparatus of claim 4 wherein the controller further includes an ICP state linearizer as part of the algorithm control scheme to maintain the ICP close to or on a desired clinical path.
